(19) United States
(12) Patent Application Publication
(10) Pub. No.: US 2013/0142739 A1
(43) Pub. Date: Jun. 6, 2013

(54) PROCESS FOR THE SYNTHESIS OF ISOTHIOCYANATES AND DERIVATIVES THEREOF AND USES OF SAME

(71) Applicants: Jacques Dubois, Villers-la-ville (BE); Alfred Marchal, Waterloo (BE); Damien Lacroix, Ernage (BE); Jérôme Cabou, Saint Aybert (FR)

(72) Inventors: Jacques Dubois, Villers-la-ville (BE); Alfred Marchal, Waterloo (BE); Damien Lacroix, Ernage (BE); Jérôme Cabou, Saint Aybert (FR)

(73) Assignee: Auriga International, Waterloo (BE)

(21) Appl. No.: 13/811,600

(22) PCT Filed: Jul. 20, 2011
(86) PCT No.: PCT/EP11/62477

§ 371 Date: Feb. 14, 2013

(30) Foreign Application Priority Data
Jul. 23, 2010 (BE) 2010/0456

(51) Int. Cl. C07C 331/24; C07C 331/20

(52) U.S. Cl. CPC C07C 331/24; C07C 331/20
USPC 424/59; 558/18; 424/62; 514/514

(57) ABSTRACT

A method for synthesizing an isothiocyanate of general formula (I)

SCN—R₁—R₄—R₂ (I)

wherein R₁ and R₂ represent independently of each other an alkyl-aryl or aryl group, R₄ represents a carbonyl, sulfinyl, sulfonyl group or a sulfide group and of its derivatives comprising a step for reacting an alkylalkylamine having the general formula (II)

NH₂—R₁—R₄—R₂ (II)

wherein R₁ and R₂ represent independently of each other an alkyl, aryl or alkylaryl group, R₄ represents a carbonyl, sulfinyl, sulfonyl or sulfide group, in the presence of carbon sulfide and of di-tert-butyl dicarbonate with formation of the corresponding aforesaid isothiocyanate, compounds obtained by this method as well as their uses.

The present invention relates to a method for synthesizing an isothiocyanate of general formula (I)

SCN—R₁—R₄—R₂ (I)

wherein R₁ and R₂ represent independently of each other, an alkyl, aryl or alkylaryl, R₄ represents a carbonyl, sulfonyl, sulfinyl or sulfide group, and of its derivatives.

More particularly the present invention relates to a method for synthesizing sulforaphane, a particular isothiocyanate according to general formula (I) wherein R₁ represents a butyl group, R₂ represents a methyl group and R₄ represents a sulfinyl group and of its derivatives.

Sulforaphane therefore has the following general formula (A)

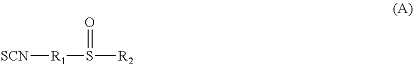

wherein R₁ represents a butyl group, R₂ represents a methyl group.

The present invention also relates to the making of analogs of sulforaphane such as 6-isothiocyanatohexan-2-one which has formula (B) i.e. a particular isothiocyanate according to general formula (I) wherein R₁ represents a butyl group, R₂ represents a methyl group and R₄ represents a carbonyl group, and of its derivatives such as thioureas obtained from sulforaphane or from 6-isothiocyanatohexan-2-one, coupling products with alcohols or thiols and sulforaphane and/or 6-isothiocyanatohexan-2-one.

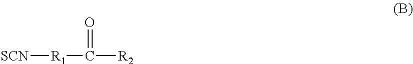

Sulforaphane is a molecule naturally present in cruciferous vegetables, such as broccoli or Brussel sprouts. This molecule aroused great interest in the 1990's since the origin of beneficial effects on health by consuming these vegetables, is ascribed to it, notably as an anti-cancer agent.

To this day, many studies have been conducted with this molecule in the field of protecting cells against aggressions and cancers, however various limitations have not yet been removed and seem to have prevented any development towards large scale studies or any commercial application.

Indeed, sulforaphane is only available on the market at high prices, this stems from the fact that the molecule has to be extracted from plants (with a lot of loss by degradation), that the few described synthesis methods have low to moderate yields and are not reproducible. Further, the molecule is unstable and therefore difficult to incorporate into a cosmetic or pharmaceutical preparation.

A method for the synthesis of alkylsulfinylalkylamine, in particular 4-methylsulfinylbutylamine as well as synthetic sulforaphane has been described in document WO 02/58664.

In this document, 4-chlorobutyronitrile is dissolved in absolute ethyl alcohol distilled beforehand on sodium, and methane thioate is then added. The obtained suspension is finally filtered, the filtrate is concentrated and taken up in ethyl ether. The etherated solution containing crude 4-methylthiobutyronitrile is then obtained and engaged in other steps.

In this synthesis step, the solution of 4-methylthiobutyronitrile is added to a suspension of lithium-aluminum hydride in order to obtain methylthiobutylamine. Methylthiobutylamine is then oxidized with hydrogen peroxide in the presence of acetone in order to obtain the precursor of sulforaphane, i.e. 4-methylsulfinylbutylamine, for one night at 50° C.

In a subsequent phase of the method according to document WO 02/058664, 4-methylsulfinylbutylamine reacts in the presence of thiophosgen and NaOH in order to form D,L-sulforaphane.

Unfortunately, the yields of this synthesis route are not very high on the one hand since the steps used often lead to the formation of bothersome byproducts for the subsequent steps, which requires intermediate purification steps having a negative effect on the overall yields. Indeed, document WO 02/508664 describes a yield of 31% for the step of generating the isothiocyanate combined with a distillation step, which leads to an overall yield of 18% on the whole synthesis method described in document WO 02/508664. On the other hand, the reagents used in this known synthesis route are all expensive, noxious for the environment or toxic.

Therefore there exists a real need for producing a sulforaphane or an analog of the latter, such as for example in the form of 6-isothiocyanatohexan-2-one, as well as their derivatives in a clean, non-toxic way and which has good yields.

For this purpose, the method according to the invention provides a method as mentioned initially comprising a reaction of an amine having the general formula (II)

NH₂—R₁—R₄—R₂ (II)

wherein R₁ and R₂, represent independently of each other an alkyl, aryl or alkylaryl group, R₄ represents a carbonyl, sulfinyl, sulfonyl or sulfide group in the presence of carbon sulfide and of di-tert-butyl dicarbonate with formation of the aforesaid isothiocyanate corresponding to general formula (I).

In this way, isothiocyanate compounds according to general formula (I), such as for example sulforaphane or 6-isothiocyanatohexan-2-one are synthesized from their corresponding amine while avoiding the use of thiophosgen which is a particularly toxic substance and without using “thiocarbonyl transfer” reactions conducted with “substituents” of thiophosgen such as thiocarbonylditriazole or thiocarbonyldiimadozle or further dipyridyl-thionocarbonate. These substituents may actually be efficient but they are not economical in terms of atoms and therefore generate byproducts which are not always easy to remove at the end of synthesis. Such methods will therefore require an additional step such as chromatographic distillation or purification which is detrimental to the yield, to the production costs and do not always give satisfactory purity, especially taking into account the fact that sulforaphane degrades with heat.

In the method according to the invention, the use of carbon sulfide CS₂ and of di-tert-butyl dicarbonate (BOC₂O) only generates byproducts which are simple and easy to remove at a lesser cost and therefore without any incidence on the yield on the method according to the invention and on the environment. Indeed, the byproducts generated by the reaction of the method according to the invention are: CO₂; COS; tBuOH.

As mentioned earlier, one of the goals according to the invention is to provide in a clean and inexpensive way sulforaphane or 6-isothiocyanatohexan-2-one and their derivatives or analogs such as, for example, sulforamate with view to having a sufficient amount available for studying the potential effects of the latter in cosmetics or in cancer treatments.

Advantageously, in the method according to the invention, R₄ represents a sulfinyl group and said amine is an alkylsulfinylalkylamine. More particularly, said amine of general formula (I) is 4-methylsulfinylbutylamine and said corresponding formed isothiocyanate is sulforaphane.

In an advantageous embodiment of the method according to the invention, said alkylsulfinylalkylamine is obtained by oxidation of alkylthioalkylamine in solution in a solvent based on trifluoroethanol.

As this may be noticed from the foregoing, the method according to the invention uses trifluoroethanol as a solvent for achieving oxidation of the alkylthioalkylamine into an alkylsulfinylalkylamine. The use of trifluoroethanol as a solvent allows faster kinetics (about 30 mins at 0° C. after adding the oxidant and then 1 hour at room temperature). This period is much shorter than that of document WO 02/58664 (one night at 50° C. with acetone as a solvent). Further according to the invention, the obtained product is pure and does not have sulfinyl traces, which means that the oxidation is under control and does not result in the formation of over-oxidized products mixed with the thereby produced sulfinyl.

Further, according to the invention, the oxidation product is obtained in an isolated way and is pure at the end of the step, which allows some flexibility lying in the fact of linking up other steps or storing the product in this form. In any case, the yield is better since it is not necessary to link up a multitude of purification steps in order to obtain the product.

In this way, the use of acetone customarily used in syntheses of this type or that of sulforaphane is avoided by the use of trifluoroethanol as a medium for the oxidizing agent. This provides better control of the reaction (no over-oxidation) which improves the yield.

This synthesis step is therefore clean and therefore does not generate any over-oxidation products, the fluorinated solvent which is more expensive than a standard solvent will advantageously be recovered, by simple distillation in order to further reduce the costs of the method according to the invention.

In an alternative according to the invention, R₄ represents a carbonyl group and said amine comprises a keto group.

More particularly, said amine comprising a keto group is 4-methylketobutylamine or 6-isothiocyanatohexan-2-one and the corresponding isothiocyanate formed is 6-isothiocyanatohexan-2-one.

In a preferential embodiment according to the invention, the method also comprises a reaction of said corresponding isothiocyanate such as for example sulforaphane or its analog 6-isothiocyanatohexan-2-one, of general formula (I) with a primary or secondary amine in order to form a thiourea given as an example, derived from sulforaphane or from 6-isothiocyanatohexan-2-one.

Actually, it is possible to contemplate the coupling of a wide panel of amines selected as reference molecules for these syntheses, since they are assumed to have a depigmenting effect which may reinforce the depigmenting effect of sulforaphane as well as the potential one of 6-isothiocyanatohexan-2-one, both isothiocyanates.

In general, in humans, isothiocyanates are rapidly absorbed and then combine with thiols of proteins, cysteine or glutathione. In the body this combination reaction is a reversible process and the adduct may dissociate to release the isothiocyanate again.

When the isothiocyanates combine with glutathione, they are then metabolized through the route of mercapturic acids so as to be finally excreted in urines mainly as adducts of N-acetyl cysteine.

As described above, it is therefore the isothiocyanate function which is involved in the metabolization of sulforaphane. It notably reacts with glutathione and the thiols of amino acids, for this reason, provision may be made for 6-isothiocyanatohexan-2-acne to represent an interesting analog, given its close structure and the presence of the isothiocyanate radical.

It is notably by means of this type of interactions that sulforaphane may induce many effects which are assigned to it (for example: stimulation of enzymes of phase II, inhibition of AP1 a transcription factor involved in skin cancers by interaction with sulforaphane and thiols of cysteines, . . . ).

Moreover, sulforaphane is accumulated in cells in its form conjugate to glutathione. Indeed, the major portion of the intracellular forms of isothiocyanates are dithiocarbamate forms resulting from the reaction of the isothiocyanate on glutathione. Thus, 95% of the product accumulated in the cells is in this form.

Further, it was demonstrated that although the dithiocarbamate form is the form for storing sulforaphane at the cell (rapid accumulation in high concentrations), the cells are not capable of directly absorbing this form if it is already presented to them in a pre-shaped form instead and in place of the corresponding isothiocyanate. In this case, there would in a first phase be extracellular hydrolysis of the dithiocarbamate in order to return to the free isothiocyanate, a form which may then be integrated by the cell and then converted for storage in the form of dithiocarbamate.

The rapid accumulation with high concentrations of these isothiocyanates stored in the form of dithiocarbamate, seems to play a crucial role in their capability of inducing the system of enzymes of phase II and notably an anti-cancer protective effect.

From what is mentioned above, it emerges that the derivatives of sulforaphane and potentially those of 6-isothiocyanatohexan-2-one, such as the coupling product with glutathione, may not be directly stored by the cell but are first cleaved in vivo in order to return to sulforaphane or to 6-isothiocyanatohexan-2-one which is phagocytized by the cells, in order to be then stored as glutathione.

Thus, the amine-sulforaphane or amine-6-isothiocyanato-hexan-2-one coupled structures according to the present invention would also be cleaved in humans during metabolization, which would cause release of sulforaphane with a depigmenting effect (or of 6-isothiocyanatohexan-2-one) on the one hand and of the free amine on the other hand which would also play its role of depigmenting agent. Consequently, these derivatives of sulforaphane or of 6-isothiocyanatohexan-2-one allow several possibilities of action: either the thiourea is active per se, or it is metabolized in order to release two molecules each having a depigmenting effect or potentially a depigmenting effect.

Of course what has been described here for the depigmenting effect is also applied against hyperpigmentation, to sunscreen protection, to protection against radiations, as an anti-cancer effect and other effects mentioned above. According to the invention, the selected amine will depend on the desired effect for this coupling compounds.

For example, it is also possible to contemplate e.g. the coupling of known amines in order to have a UV filtering effect (for example: para-aminobenzoic acid or anthranilate) or further the coupling with diamines such as piperazine in order to obtain a molecule having two thiourea functions with 2 “sulforaphane” or “6-isothiocyanatohexan-2-one” units or a central diamine.

In one advantageous embodiment of the method according to the invention, the amine is a primary amine having the general formula HNR₅R₆ wherein R₅ represents a hydrogen atom and R₆ a methylsulfinylbutyl group. With this, it is possible to obtain 1,3-bis-(4-(methylsulfinyl)butyl)-thiourea.

This derivative of sulforaphane is i.a. described in patent application WO 02/58664. According to this document, the synthesis of this thiourea uses the degradation process of sulforaphane in water (degradation of sulforaphane into 4-methylsulfinylbutylamine and it is the latter which reacts on sulforaphane still present in order to form the thiourea. According to the invention, the object is to attain this thiourea in a controlled way and with a high yield, for example from the 2 pure reagents in a suitable solvent. Thus, the reaction is conducted at a low temperature (45° C. instead of 100° C. and within a shorter time (1 hour instead of 24 hours)) and the obtained yield is about 97%, with which a pure product may be obtained, which may be used for subsequent analyses.

In an alternative according to the invention, the amine is a primary amine having the general formula HNR₅R₅ wherein R₅ represents a hydrogen atom and R₆ a linear, cyclic or branched alkyl, alkenyl, alkylaryl, aryl, alkynyl group optionally including one or more heteroatoms.

In another alternative of the invention, the amine is a secondary amine having the general formula HNR₅R₆ wherein R₅ and R₆ represent independently of each other a linear, cyclic or branched alky, alkenyl, alkyaryl, aryl, alkynyl group optionally including one or more heteroatoms.

In an alternative embodiment according to the invention, the analogue of sulforaphane or of 6-isothiocyanatohexan-2-one is a derivative of the latter formed by a reaction between sulforaphane or 6-isothiocyanatohexan-2-one and a nucleophilic agent, in particular an alcohol or a thiol and preferably ethyl alcohol.

With the reaction between sulforaphane and a thiol, such as for example with the methanethiol generated in situ from sodium methanethiolate it is possible to obtain another derivative of sulforaphane, the sulforamate. The synthesis is described in more detail in the examples below.

The reaction between 6-isothiocyanatohexan-2-one and a thiol, such as for example with methanethiol generated in situ from sodium methanethiolate gives the possibility of obtaining the carbonyl equivalent of the sulforamate, methyl-5-oxohexylcarbamodithioate. The synthesis is described in more detail in the examples below.

In another embodiment, the method according to the invention comprises oxidation of the sulfinyl radical into a sulfonyl radical, by addition of an oxidizing agent, in particular a perbenzoic acid or one of its halogenated derivatives, in particular metachloroperbenzoic acid. This oxidation may take place either on the sulforaphane directly before the reaction of addition of an amine or of a nucleophilic agent, or directly on the formed analog.

Other embodiments of the method according to the invention are indicated in the appended claims.

The object of the invention is also a synthetic and isolated compound of general formula (Ill) obtained by the method according to the invention

R₇—R₁—R₄—R₂ (III)

wherein R₄ is a carbonyl, sulfinyl or sulfonyl or sulfide group, R₇ represents an amino-isiothiocyanato group, a group of the —NH—C(═S)—R′ type wherein R′ is of the alcoholate (-DR″), thiolate (SR″), amino (—NR″R′″) type and R₁ and R₂ represent independently of each other an alkyl, aryl, or alkylaryl group.

In particular, the invention relates to the particular compounds:
to the synthetic sulforaphane obtained by the method according to the invention, i.e. to the compound of general formula (III) wherein R₄ is a sulfinyl group and R₇ represents an isothiocyanato group, R₁ represents a butyl group and R₂ represents a methyl group;
to the synthetic 6-isothiocyanatohexan-2-one obtained by the method according to the invention, i.e. to the compound of general formula (III) wherein R₄ is a carbonyl group and R₇ represents an isothiocyanato group, R₁ represents a butyl group and R₂ represents a methyl group;
to the synthetic thioureas obtained by the method according to the invention, in particular synthetic 1,3-bis-(4-(methylsulfinyl)butyl)-thiourea, N-4-(methylsulfinyl)butyl)piperidine-1-carbothioamide, 4-methyl-N-4-(methylsulfinyl)butyl)piperazine-1-carbothioamide, N-4-(methylsulfinyl)-butyl)morpholine-4-carbothioamide, 1-(benzylpiperidin-4-yl)-3-(4-methyl-sulfinyl)butyl)thiourea obtained by the method according to the invention.

Further, the present invention also relates to derivatives of formula (III) wherein R₂ is for example, without however being limited thereto, an O-ethyl-carbamothioate group i.e. wherein R₇ is —NH—C(═S)—R′ and R′ represents OH, obtained by the method according to the invention, i.e by coupling an alcohol (for example ethanol) on sulforaphane, to the oxidized sulforaphane (4-methylsulfonyl)butylisothiocyanate; R₄ then being a sulfinyl group),
to the thioureas obtained by coupling of primary or secondary amines on oxidized sulforaphane (such as for example 1-(4-(methylsulfinyl)butyl)-3(4-methylsulfonyl)butyl)thiourea and 1,3-bis(4-(methylsulfonyl)butyl)thiourea), to the coupling products between an alcohol (or a thiol) and oxidized sulforaphane and to precursors such as 4-methylsulfinylbutylamine,
to the thioureas obtained by coupling of primary or secondary amines on 6-isothiocyanatohexan-2-one as mentioned above.

Other embodiments of the compound according to the invention are indicated in the appended claims.

The present invention also relates to a use of the compounds according to the invention as a depigmenting agent in the treatment of hyperpigmentation, as an inducer of enzymes of phase H, as an inhibitor or moderator of enzymes of phase 1 in an anti-cancer treatment, in the protection of skin against radiations (UV and other radiation), in the treatment of canities, in the protection of skin against UVs and effects thereof, for example in phototherapies, radiotherapies, exposures to the sun at erythemas, damages to DNA, induction, repair, signalling, cancerization, in the treatment of inflammation, of atopic dermatitis, in the protection of the skin against the effects of pollution and in the treatment of lucite.

In the sense of the present invention, the groups R₁, R₂, R₃, R₅, R₆, R₇, R′, R″ and R′″ each advantageously have independently of each other from 1 to 20 carbon atoms, preferably from 1 to 18 carbon atoms, for example from 1 to 12 carbon atoms.

Other embodiments of the uses according to the invention are indicated in the appended claims.

Other features, details and advantages of the invention will become apparent from the description given hereafter, not as a limitation and referring to the non-limiting examples.

The present invention therefore relates to a method for synthesizing sulforaphane mainly comprising the successive steps for synthesizing 4-methylthiobutyronitrile (A), for synthesizing 4-methylthiobutylamine (B), for synthesizing 4-methylsulfinylbutylamine (C) and for synthesizing sulforaphane (4-methylsulfinylbutyl isothiocyanate) (D). These steps are indicated below.

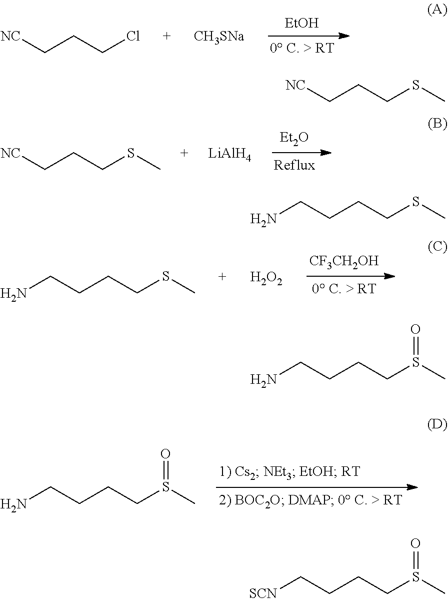

Also according to the invention once sulforaphane is formed, it may then react with amines in order to form derivatives of sulforaphane such as for example 1,3-bis-(4-(methylsulfinyl)butyl)-thiourea (A): the global reaction (E)

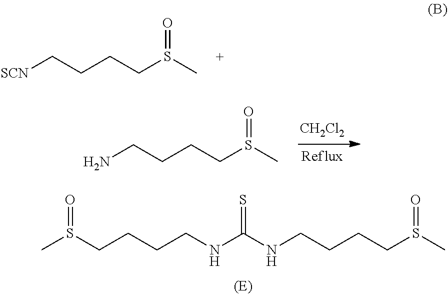

Another derivative of sulforaphane which may be produced according to the invention is a N-4-(methylsulfinyl)butyl)-piperidine-1-carbothiomide by adding an amine, piperidine. This compound has the formula (V)

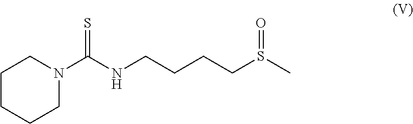 [SMILES: CS(=O)CCCCNC(=S)N1CCCCC1]

Another derivative of sulforaphane which may be produced according to the invention is N-4-(methylsulfinyl)butyl)-morpholine-4-carbothioamide by addition of an amine, morpholine. This compound has the formula (VI)

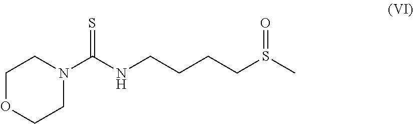 [SMILES: CS(=O)CCCCNC(=S)N1CCOCC1]

Still another derivative of sulforaphane which may be produced according to the invention is 1-(benzylpiperidin-4-yl)-3-(4-methylsulfinyl)butyl)thiourea by adding an amine, benzylpiperidine. This compound has the formula (VII).

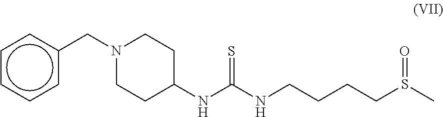

Further, as mentioned earlier, the invention also provides the preparation of certain derivatives of sulforaphane such as for example the oxidized form of the latter according to reaction (G).

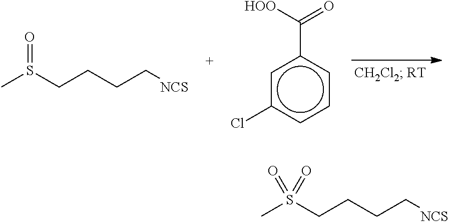

In the sense of the invention, other oxidized forms of the derivatives of sulforaphane are also obtained according to the invention in an analogous way with the one described above. Thioureas deriving from oxidized sulforaphane following the two possible cases 1-(4-(methylsulfinyl)butyl)-3(4-methylsulfonyl)butyl)thiourea having the following formula (VIII) and 1,3-bis(4-(methylsulfonyl)butyl)thiourea) having the following formula (IX). Further, other thioureas may be obtained by coupling of either cyclic or non-cyclic primary or secondary amines on oxidized sulforaphane. These amines will for example be selected according to the properties of said amines, these compounds then have the following formula (X). If the coupling of the oxidized sulforaphane is achieved with an alcohol, a compound according to the formula (XI) is obtained. The formulae (VIII to XI) are shown in Table 1.

TABLE 1

| For- | |
|---|---|
| mula | |
| No. | Product |
| (VIII) | 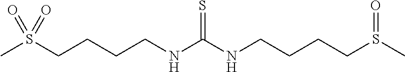 |
| (IX) | 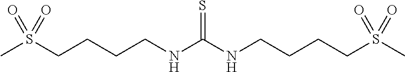 |
| (X) | 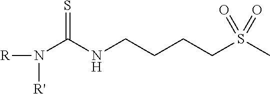 |
| (XI) | 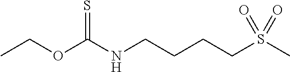 |

The present invention therefore also relates to a method for synthesizing 6-isothiocyanatohexan-2-one comprising the synthesis step (H) mentioned above.

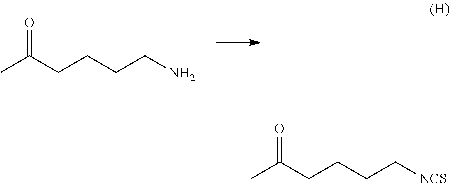

Various analogs or derivatives of 6-isothiocyanatohexan-2-one which may be produced according to the invention are mentioned below. For example, one of the derivatives which may be obtained according to the invention is 1,3-bis-(5-oxohexyl)thiourea. By degradation of 6-isothiocyanatohexan-2-one in water. This compound has the formula (XII),

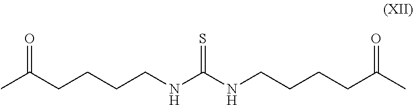 [SMILES: CC(=O)CCCCNC(=S)NCCCCC(C)=O]

For example one of the derivatives which may be obtained according to the invention is 1-(4-(methylsulfinyl)butyl)-3(5-oxohexyl)thiourea by addition of 4-methylsulfinylbutylamine. This compound has the formula (XIII).

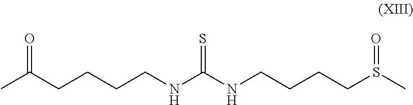 [SMILES: CC(=O)CCCCNC(=S)NCCCCS(C)=O]

For example one of the derivatives which may be obtained according to the invention is 1-(4-(methylsulfinyl)butyl)-3-(5-oxohexyl)thiourea by adding an amine, 4-methylsulfinylbutylamine. This compound has the formula (XIV).

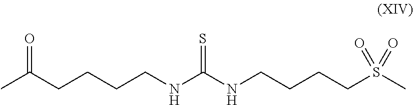 [SMILES: CC(=O)CCCCNC(=S)NCCCCS(C)(=O)=O]

For example one of the derivatives which may be obtained according to the invention is N-5-(oxohexyl)morpholine-4-carbothioamide by adding morpholine. This compound has the formula (XV).

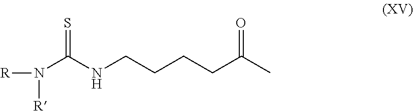

For example one of the derivatives which may be obtained according to the invention is O-ethyl-N-5-oxohexylcarbamothioate by adding an alcohol (ethanol). This compound has the formula (XVI).

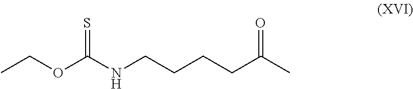

### EXAMPLE 1

### Synthesis of 4-methylthiobutyronitrile

53.38 g of 4-chlorobutyronitrile (1 equivalent) in solution in 250 mL of ethanol are placed in an isobaric dropping funnel, surmounting a 1 liter flask.

1.1 equivalents of sodium methanethiolate, as an aqueous 21% sodium methanethiolate solution (Sigma-Aldrich) are introduced into the reactor.

The flask is cooled by means of a water-ice bath and the system is degassed, and then placed under nitrogen and maintained with stirring.

The solution containing the nitrile is then poured into the reactor within about one hour (rapidly dropwise).

This time having elapsed, the ice bath is removed so as to return to room temperature. The stirring is then maintained for further 24 hours at this temperature.

At the end of the reaction, 400 mL of water are added, as well as 200 mL of dichloromethane. The solution is decanted and the aqueous phase is extracted twice again with dichloromethane. The collected organic phases are then washed with water and then dried on magnesium sulfate and filtered.

The solvent is finally removed in the rotary evaporator. 58.59 g of a colorless liquid are then recovered and engaged into the following step without requiring any additional purification. The yield is considered as quantitative.

TLC: Eluent: Hexane—Et₂O 50/50;

Developer: phosphomolybdic acid;

Rf=0.45

¹H NMR (300 MHz; CDCl₃):

δ (ppm): 2.62 (t, 2H, J=6.6 Hz, CH₂CN); 2.51 (t, 2H, J=7.0 Hz, CH₂SCH₃); 2.10 (s, 3H, CH₂SCH₃); 1.94 (m, 2H, CH₂CH₂CH₂)

¹³C NMR (75 MHz; CDCl₃):

δ (ppm): 119.1; 32.6; 24.6; 15.8; 15.3

Refractive index: n_{D}²⁰=1.4814

### EXAMPLE 2

### Synthesis of 4-methylthiobutylamine

17.26 g of 4-methylthiobutyronitrile (1 equivalent) in solution in 80 mL, of anhydrous ether are placed in an isobaric dropping funnel surmounting a 1 liter flask. 1.5 equivalents of LiAlH₄, as well as 300 of anhydrous diethyl ether are introduced into the reactor surmounted with a coolant.

The assembly is degassed, and then placed under nitrogen and maintained with stirring at room temperature.

4-methylthiobutyronitrile is then slowly poured. The addition causes heating of the medium, the addition rate is then controlled so as to control the reflux which is set up.

After addition, the medium is maintained with stirring and with reflux for 3 hours.

This time having elapsed, the reactor is cooled with a water-ice bath and then 100 mL of distilled water are slowly poured in via the dropping funnel in order to neutralize the excess of LiAlH₄.

The mixture is then filtered on a frit and the filter is washed several times with ether.

The filtrate is then decanted, the organic phase is filtered on charcoal, the filter is rinsed and the collected organic phases are dried on sodium sulfate.

The solvent is finally removed in the rotary evaporator. 18.10 g of a slightly yellow liquid are then recovered and engaged in the following step without requiring any additional purification. The yield is considered as quantitative.

TLC: IPC—Eluent: Hexane—Et₂O 50/50;

Developer: Phosphomolybdic acid

Finished product—Eluent: MeOH—5% HCOOH;

Developer: Phosphomolybdic acid or ninhydrin;

Rf=0.5

¹H NMR (300 MHz; CDCl₃):

δ (ppm): 2.70 (t, 2H, J=7.0 Hz, CH₂NH₂); 2.50 (t, 2H, J=7.0 Hz, CH₂SCH₃); 2.09 (s, 3H, CH₂SCH₃); 1.65-1.48 (m, 6H, CH₂(CH₂)₂CH₂ CH₂NH₂)

¹H NMR (300 MHz; D₂O):

δ (ppm): 2.64 (t, 2H, J=7.0 Hz, CH₂NH₂); 2.57 (t, 2H, J=7.0 Hz, CH₂SCH₃); 2.11 (s, 3H, CH₂SCH₃); 1.68-1.47 (m, 4H, CH₂(CH₂)₂CH₂)

¹³C NMR (75 MHz; CDCl₃):

δ (ppm): 41.7; 34.1; 32.8; 26.5; 15.5

¹³C NMR (75 MHz; D₂O):

δ (ppm): 40.2; 33.2; 30.8; 25.8; 14.2

Refractive index: n_{D}²⁰=1.4831

### EXAMPLE 3

### Synthesis of 4-methylsulfinylbutylamine

17.98 g of 4-methylthiobutylamine (1 equivalent) are introduced into the reactor cooled down to 0° C. The assembly is degassed and then placed under nitrogen under stirring. 75 mL of trifluoroethanol are then slowly poured in order to form the amine solution. The addition should be accomplished at 0° C. since exothermy is detected.

1.1 equivalents of hydrogen peroxide (35% in water) are then poured dropwise into the reactor, within 30 minutes, via an isobaric dropping funnel.

Once the addition is finished, the reaction medium is brought back to room temperature and maintained with stirring, at this temperature, for 1 hour.

This time having elapsed, 3 g of active charcoal are introduced into the reactor. After 20 minutes of stirring at room temperature, the reaction mixture is filtered on celite and the filter is washed with 100 mL of ethanol.

The filtrate is recovered and concentrated in the rotary evaporator at a bath temperature of 50° C. The product is taken up with 60 mL of dichloromethane and dried on MgSO₄, and the solvent is then removed with the rotary evaporator.

18.35 g of a slightly yellow liquid are thereby obtained, i.e. with a yield of 90%. The product is engaged in the following step without requiring any additional purification.

TLC: Eluent: MeOH—5% HCOOH;

Developer: phosphomolybdic acid or ninhydrin;

Rf=0.45.

¹H NMR (300 MHz; CDCl₃):

δ (ppm): 2.79-2.62 (m, 4H, —S(O)—CH₂—+CH₂—NH₂); 2.56 (s, 3H, CH₃); 1.87-1.76 (m, 2H, —CH₂—CH₂—); 1.71-1.59 (m, 2H, —CH₂—CH₂—); 1.57 (broad peak, 2H, NH₂).

¹H NMR (300 MHz; D₂O):

δ (ppm): 2.92 (m, 2H, —S(O)—CH₂—); 2.70 (s, 3H, CH₃); 2.68 (t, J=7.0 Hz, 2H, —CH₂—NH₂); 1.83-1.72 (m, 2H, —CH₂—CH₂—); 1.67-1.56 (m, 2H, —CH₂—CH₂—).

¹³C NMR (75 MHz; CDCl₃):

δ (ppm): 54.39 (—CH₂—NH₂); 41.56 (—S(O)—CH₂—); 38.52 (CH₃—); 32.56 (—CH₂—CH₂—NH₂); 19.97 (—S(O)—CH₂—CH₂—),

¹³C NMR (75 MHz; D₂O):

δ (ppm): 55.21; 42.81; 39.22; 33.29; 22.29.

Refractive index: n_{D}²⁰=1.4855

### EXAMPLE 4

### Synthesis of Sulforaphane

18.39 g of 4-methylsulfinylbutylamine (1 equivalent), as well as 200 mL of absolute ethanol are introduced into a reactor, and the mixture is then stirred at room temperature, degassed and placed under nitrogen.

1 equivalent of triethylamine is then poured into the reactor, followed by 10 equivalents of carbon disulfide. The addition of CS₂ is exothermic and has to be accomplished dropwise; the medium assumes a yellow coloration.

The thereby formed mixture is maintained with stirring at room temperature for 30 mins before being cooled to 0° C.

0.99 equivalents of di-tert-butyl dicarbonate dissolved in 100 mL of absolute ethanol are then added via an isobaric dropping funnel within about 30 mins. By this same means, addition of 3 mol % of DMAP in solution in 50 mL of absolute ethanol follows.

The reaction medium is then maintained with stirring and at 0° C. for 15 mins, and then the ice bath is removed and the reaction is continued for 2 hours at room temperature.

The reaction crude mixture is transferred into a 1-neck flask and concentrated in the rotary evaporator and then taken up in 30 mL of dichloromethane in order to be filtered on charcoal. The filter is rinsed with dichloromethane and the collected filtrates are again placed in the rotary evaporator. 23.1 g of an orange oil are thereby obtained.

The raw product essentially contains traces of residual DMAP. Purification on a chromatography column was initially considered but we may advantageously replace this by simple washing.

This crude product is then taken up in 100 mL of dichloromethane in order to carry out acid washing. The obtained solution is then briefly stirred in the presence of 200 mL of 1N HCl. After decantation, the aqueous phase is again extracted once with dichloromethane, and the collected organic phases are washed with water, dried on MgSO₄. The solvent is finally removed in the rotary evaporator. 21.12 g of a yellow (oily) liquid are thereby obtained, i.e. a yield of 88%.

The overall yield of the reaction in 4 steps is therefore 79% which to our knowledge is well above all the yields described in the literature for the various routes for accessing sulforaphane (conventionally about 7-50%).

TLC: Eluent: AcOEt-MeOH 9/1;

Developer: phosphomolybdic acid;

Rf=0.3.

¹H NMR (300 MHz; CDCl₃):

δ (ppm): 3.60 (t, 2H, J=6.1 Hz, —CH₂NCS); 2.73 (m, 2H, —S(O)—CH₂—); 2.50 (s, 3H, CH₃); 1.82-2.00 (broad m, 4H, —CH₂—CH₂—).

¹³C NMR (75 MHz; CDCl₃):

δ (ppm): 53.48 (—CH₂—NCS); 44.62 (—S(O)—CH₂); 38.71 (CH₃—S(O)—); 28.97 (—CH₂—CH₂—NCS); 20.067 (—S(O)—CH₂—CH₂—).

UV spectrum:

λₘₐₓ=242 nm.

Refractive index: n_{D}²⁰=1.3516

### EXAMPLE 5

### Synthesis of an Analog of Sulforaphane, 1-(ethylsulfinyl)-4-isothiocyanatobutane isothiocyanate

The procedure of Examples 1 to 4 is reproduced except from the fact that the methanethiolate of Example 1 was replaced with sodium ethanethiolate in order to obtain the compound, 1-(ethylsulfinyl)-4-isothiocyanatobutane illustrated by the following formula

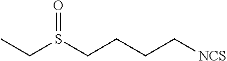

This isothiocyanate is synthesized according to the same method as the sulforaphane of Example 4, by reaction of the corresponding amine (4-(ethylsulfinyl)butanamine) in the presence of carbon disulfide and of di-tert-butyl dicarbonate. The amine is obtained by following the first three steps for the synthesis of sulforaphane described in this patent, by simply replacing CH₃SNa by EtSNa in the first step.

4-(ethylsulfinyl)butanamine reacts in ethanol in the presence of 1 equivalent of triethylamine and of 10 equivalents of carbon disulfide. After reaction the mixture is cooled to 0° C. before adding thereto 0.99 equivalents of di-tert-butyl dicarbonate and 3 mol % of DMAP in solution in ethanol. After returning to room temperature and a further 2 hours of reaction, the reaction medium may be treated: removal of the solvent, washing with a solution of hydrochloric acid, decantation and removal of the solvent. The isothiocyanate is obtained as a yellow liquid which solidifies while cooling with a yield of 87% for this step.

The overall yield of the reaction, including synthesis of the amine, is 87%.

TLC:

Eluent: EtOAc MeOH 90/10; Developer: phosphomolybdic acid;

Rf=0.4

¹H NMR (300 MHz; CDCl₃):

δ (ppm): 3.57 (t, 2H, J=6.2 Hz, CH₂NCS); 2.68 (m, 4H, CH₂—(S═O)—CH₂); 1.91 (m, 4H, —CH₂—CH₂—); 1.32 (t, 2H, J=7.5 Hz, CH₃CH₂—(S═O)—)

¹³C NMR (75 MHz; CDCl₃):

δ (ppm): 50.44; 45.79; 44.57; 28.98; 20.07; 6.69

Melting point: 44° C.

UV SPECTRUM

λₘₐₓ=245 nm

### EXAMPLE 6

### Synthesis of an Analog of Sulforaphane, (4-isothiocyanatobutylsulfinyl)benzene isothiocyanate

The procedure of Examples 1 to 4 is reproduced except for the fact that sodium methanethiolate according to Example 1 has been replaced with sodium thiophenolate in order to obtain the compound (4-isothiocyanatobutylsulfinyl)benzene illustrated by the following formula,

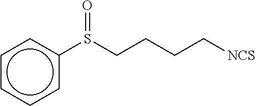

This isothiocyanate is synthesized according to the same method as for sulforaphane by reaction of the corresponding amine (4-phenylsulfinyl)butanamine) in the presence of carbon disulfide and di-tert-butyl dicarbonate. The amine is obtained by following the first three synthesis steps of sulforaphane described in this patent, simply by replacing CH₃SNa with PhSNa in the first step.

4-(phenylsulfinyl)butanamine reacts, in ethanol, in the presence of 1 equivalent of triethylamine and of 10 equivalents of carbon disulfide. After reaction, the mixture is cooled to 0*C before adding thereto 0.99 equivalents of di-tert-butyl dicarbonate and 3 mol % of DMAP in solution in ethanol. After returning to room temperature and a further 2 hours of reaction, the reaction medium may be treated: removal of the solvent, washing with a hydrochloric acid solution, decantation and removal of the solvent. The isothiocyanate is obtained as a very viscous yellow oil with a yield of 96%.

The overall yield of the reaction, including the synthesis of the amine, is 57%.

TLC:

Eluent: EtOAc—petroleum ether 2/1; Developer: phosphomolybdic acid; Rf=0.2

¹H NMR (300 MHz; CDCl₃):

δ (ppm): 7.47-7.61 (m, 5H, Ph); 3.51 (m, 2H, CH₂NCS); 2.79 (m, 2H, CH₂SPh); 1.84 (m, 4H, —CH₂CH₂)

¹³C NMR (75 MHz; CDCl₃):

δ (ppm): 143.30; 131.01; 129.22; 123.81; 55.79; 44.48; 28.82; 19.40

UV SPECTRUM

λₘₐₓ=241 nm

### EXAMPLE 7

### Synthesis of 1,3-bis-(4-(phenylsulfinyl)-butyl)-thiourea from (4-isothiocyanato-butylsulfinyl)benzene Obtained in Example 6

The (4-isothiocyanatobutylsulfinyl)benzene of Example 6 is placed in a reactor in the presence of water, and then the mixture is refluxed and maintained at this temperature for 24 hours. After removing the solvent, the reaction crude mixture is purified on a silica gel chromatographic column (CH₂Cl₂/MeOH). The thiourea is thereby obtained pure in the form of a viscous oil with a yield of 94% and has the following formula,

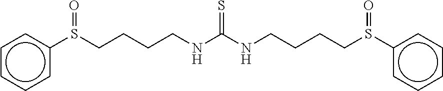

TLC:

Eluent: CH₂Cl₂—MeOH 9/1; Developer: phosphomolybdic acid;

Rf=0.7

¹H NMR (300 MHz; CDCl₃):

δ (ppm): 7.49-7.60 (m, 10H, Ph); 6.78 (broad peak, 2H, —NH—); 3.49 (m, 4H, —CH₂NH—); 2.80 (m, 4H, —CH₂(S═O)—); 1.65-1.85 (m, 8H, —CH₂—CH₂—CH₂—CH₂)

¹³C NMR (75 MHz; CDCl₃):

δ (ppm): 143.47; 131.13; 129.31; 123.87; 56.37; 43.47; 27.97; 19.82

UV SPECTRUM

λₘₐₓ=243 nm

### EXAMPLE 8

### Synthesis of the Isothiocyanate; 5-methylsulfinylpentyl isothiocyanate

The procedure of Examples 1 to 4 is reproduced except for the fact that the 4-chlorobutyronitrile of Example 1 was replaced by 4-chloropentanenitrile in order to obtain the compound: 5-methylsulfinylpentyl isothiocyanate illustrated by the following formula:

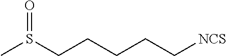

This isothiocyanate is synthesized according to the same method as sulforaphane, by reaction of the corresponding amine, (5-(methylsulfinyl)pentanamine) in the presence of carbon disulfide and of di-tert-butyl Bicarbonate. The amine is obtained by following the first three steps of synthesis of sulforaphane described in this patent, simply by replacing the nitrile by its homolog.

The 5-(methylsulfinyl)pentanamine reacts in ethanol in the presence of 1 equivalent of triethylamine and of 10 equivalents of carbon disulfide. After reaction, the mixture is cooled to 0° C. before adding thereto 0.99 equivalents of di-tert-butyl dicarbonate and 3 mol % of DMAP in solution in ethanol. After returning to room temperature and a further 2 hours of reaction, the reaction medium may be treated: removal of the solvent, washing with a hydrochloric acid solution, decantation and removal of the solvent. The isothiocyanate is obtained as a yellow liquid with a yield of 88% for this step.

The overall yield of the reaction, including synthesis of the amine, is 86%.

TLC: Eluent: EtOAc MeOH 90/10; Developer: phosphomolybdic acid;

Rf=0.3

¹H NMR (300 MHz; CDCl₃):

δ (ppm): 3.51 (t, 2H, J=6.4 Hz, —CH₂CN); 2.661 (m, 2H, —CH₂(S═O)—; 2.53 (s, 3H, CH₃—(S═O)—); 1.51-1.61 (m, 4H, —CH₂CH₂CH₂—); 1.67-1.83 (m, 2H, —CH₂CH₂CH₂—)

¹³C NMR (75 MHz; CDCl₃):

δ (ppm): 54.03; 44.57; 38.52; 29.40; 25.66; 21.77

Refractive index: n_{D}²⁰=1.5518

UV SPECTRUM

λₘₐₓ=245 nm

### EXAMPLE 9

### Synthesis of 1,3-bis-(4-methlsulfinyl)-butyl)-thiourea

4.43 g of sulforaphane obtained in Example 4 (1 equivalent), are dissolved in 10 mL of dichloromethane and placed in an isobaric dropping funnel surmounting a reactor in which are placed 4.79 g of 4-methylsulfinylbutylamine (1.1 equivalents), as well as 40 mL of dichloromethane.

The mixture is refluxed and maintained thus, with stirring for 1 hour. The solvent is then removed in the rotary evaporator, and the crude mixture is then taken up in dichloromethane added with hexane in order to obtain precipitation of the thiourea. The thereby obtained solid is milled, and then successively washed with diethyl ether and with hexane before being dried. 7.6 g of a white powder are finally obtained, i.e. a yield of 97%.

TLC: Eluent: CH₂Cl₂—MeOH 8/2;

Developer: phosphomolybdic acid or ninhydrin;

¹H NMR (300 MHz; CDCl₃):

δ (ppm): 6.91 (broad s, 2H, —NH—); 3.54 (m, 4H, —CH₂—NH—); 2.74 (t, J=7.2 Hz, 4H, S(O)—CH₂—); 2.57 (s, 6H, CH₃—S(O)—); 1.70-1.87 (m, 8H, —CH₂—CH₂—).

¹³C NMR (75 MHz; CDCl₃):

δ (ppm): 182.51; 53.60; 43.32; 38.49; 28.09; 19.98.

UV spectrum:

λₘₐₓ=239 nm

Melting point; 89° C.

### EXAMPLE 10

### Synthesis of N-(4-(methylsulfinyl)butyl)-piperidine-1-carbothioamide

N-(4-methylsulfinyl)butyl)piperidine-1-carbothioamide was synthesized by refluxing with heating for 1 hour in dichloromethane, 1.1 equivalents of piperidine in the presence of 1 equivalent of sulforaphane obtained in Example 4. At the end of the reaction, the solvent is evaporated. The thiourea was then crystallized as a yellowish solid and the amine excess is removed in the wash waters of the solid. The yield is 90% for the final step for coupling the amine on the sulforaphane.

¹H NMR (300 MHz; CDCl₃):

δ (ppm): 6.16 (broad peak, 1H, —NH—); 3.75 (t, J=5.3 Hz, 4H, —CH₂—N—CH₂; piperidine); 3.70 (m, 2H, —CH₂—NH—C(S)—); 2.73 (t, J=7.4 Hz, 2H, —CH₂—S(O)—); 2.54 (s, 3H, CH₃—S(O)—); 1.75-1.88 (m, 4H, —CH₂—CH₂—); 1.67-1.51 (m, 6H, —CH₂—CH₂—CH₂-piperidine).

¹³C NMR (75 MHz; CDCl₃):

δ (ppm): 180.94; 53.36; 48.74; 44.94; 38.55; 28.00; 25.39; 24.20; 19.73.

UV spectrum:

λₘₐₓ=218 and 242 nm

Melting point: 92° C.

### EXAMPLE 11

### Synthesis of 4-methyl-N-(4-(methylsulfinyl)-butyl)piperazine-1-carbothioamide

4-methyl-N-(4-(methylsulfinyl)butyl)piperazine-1-carbothioamide was synthesized by refluxing with heating for 1 hour in dichloromethane, 1.1 equivalents of piperazine in the presence of 1 equivalent of sulforaphane obtained in Example 4. At the end of the reaction, the solvent is evaporated. The thiourea was then recovered as a viscous oil on a silica gel chromatographic column and the yield is 97% for the final step for coupling the amine on the sulforaphane,

4-methyl-N-(4-(methylsulfinyl)butyl)piperazine-1-carbothioamide has the following formula:

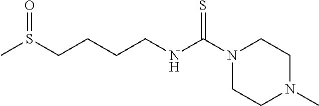

¹H NMR (300 MHz; CDCl₃):

δ (ppm): 6.24 (broad peak, 1H, —NH—); 3.84 (t, J=4.9 Hz, 4H, —CH₂—N—CH₂-ring); 3.74 (m, 2H, —CH₂—NH—C(S)—); 2.75 (1, J=7.0 Hz, 2H, —CH₂S(O)—); 2.57 (s, 3H, CH₃—S(O)—); 2.43 (t, J=5.1 Hz, 4H, —CH₂—N(Me)—CH₂-ring); 2.30 (s, 3H, CH₃—N ring); 1.93-1.83 (m, 4H, —CH₂—CH₂—).

¹³C NMR (75 MHz; CDCl₃):

δ (ppm): 181.91; 54.45; 52.99; 47.30; 45.79; 45.03; 38.61; 27.67; 19.79.

UV spectrum:

λₘₐₓ=217 and 242 nm

Crystallization/recrystallization of 4-methyl-N-(4-(methylsulfinyl)-butyl)-piperazine-1-carbothioamide from ethyl acetate is advantageous and allows the compound to be obtained as a solid in which the melting point is 72° C.

### EXAMPLE 12

### Synthesis of N-(4-(methylsulfinyl)butyl)-morpholine-4-carbothioamide

N-(4-(methylsulfinyl)butyl)morpholine-4-carbothioamide was synthesized by refluxing with heating for 1 hour in dichloromethane, 1.1 equivalents of morpholine in the presence of 1 equivalent of sulforaphane obtained in Example 4. At the end of the reaction, the solvent is evaporated. The thiourea was then crystallized as a white solid and the amine excess is removed in the wash waters from the solid. The yield is 95% for the final step for coupling the amine on the sulforaphane.

¹H NMR (300 MHz; CDCl₃):

δ (ppm): 6.46 (broad peak, 1H, —NH—); 3.83-3.69 (m, 10H, —CH₂—N—CH₂— ring +—CH₂—O—CH₂— ring +—CH₂—NH—); 2.76 (t, J=7.0 Hz, 2H, —CH₂—S(O)—); 2.57 (s, 3H, CH₃—S(O)—); 1.96-1.81 (m, 4H, —CH₂—CH₂—).

¹³C NMR (75 MHz; CDCl₃):

δ (ppm): 182.43; 66.22; 52.84; 47.55; 44.97; 38.61; 27.49; 19.76.

UV spectrum:

λₘₐₓ=218 and 242 nm

Melting point: 101° C.

### EXAMPLE 13

### Synthesis of 1-(1-benzyl-piperidin-4-yl)-3-4-(methylsulfinyl)butyl thiourea

1-(1-benzylpiperidin-4-yl)-3-4-(methylsulfinyl)butyl thiourea was synthesized by refluxing with heating for 1 hour in dichloromethane, 1.1 equivalents of benzylpiperidine in the presence of 1 equivalent of sulforaphane obtained in Example 4. At the end of the reaction, the solvent is evaporated. The thiourea was then recovered as a very viscous oil which solidifies in the refrigerator on a silica gel chromatographic column and the yield is 93% for the final step for coupling the amine on the sulforaphane.

¹H NMR (300 MHz; CDCl₃):

δ (ppm): 6.65 (broad peak, 1H, —NH—); 6.34 (broad peak, 1H, —NH—); 4.06 (broad peak, 1H, —NH—CH—); 3.56 (broad bulk, 2H, —CH₂—NH—); 3.48 (s, 2H, —CH₂-ph); 2.71-2.82 (m, 4H, —CH₂—S(O)— piperidine ring); 2.58 (s, 3H, CH₃—S(O)—); 2.18-1.42 (bulk of 4 multiplets, 10H, —CH₂—CH₂— piperidine ring).

¹³C NMR (75 MHz; CDCl₃):

δ (ppm): 181.33; 138.16; 129.07; 128.15; 126.97; 63.00; 53.48; 52.11; 51.04; 43.41; 38.55; 32.04; 28.00; 20.13.

UV spectrum:

λₘₐₓ=243 nm

Melting point: 120° C.

### EXAMPLE 14

### Synthesis of the Coupling Product of Sulforaphane with Ethanol

The sulforaphane obtained in Example 4 is solubilized in absolute ethanol. After degassing and setting it under nitrogen, the mixture is refluxed until total conversion. The ethanol is finally removed in the rotary evaporator. A viscous yellow liquid is obtained, it is purified on a chromatographic column in order to provide a beige solid with a yield of 84%.

¹H NMR (300 MHz; CDCl₃):

Like for sulforamate, the presence of a minority tautomer is detected (66° A)/33%).

### O-ethyl 4-(methylsulfinyl)butylcarbamothioate

δ (ppm): 6.65 (broad peak, 1H, —NH—); 4.45 (q, J=7.1 Hz, 2H, —O—CH₂—CH₃); 3.60 (q, J=6.6 Hz, 2H, CH₂—NH—C(S)—); 2.75 (m, 2H, —CH₂—S(O)—); 2.57 (s, 3H, —CH₃—S(O)—); 1.86-1.79 (m, 4H, —CH₂—CH₂—); 1.29 (t, J=7.1 Hz, 3H, CH₃—CH₂—O—)

Minority Tautomer:

δ (ppm): 7.10 (broad peak, 1H, —NH—); 4.53 (q, J=7.1 Hz, 2H, —O—CH₂—CH₃); 3.31 (q, J=6.5 Hz, 2H, CH₂—NH—C(S)—); 2.72 (m, 2H, —CH₂—S(O)—); 2.57 (s, 3H, —CH₃—S(O)—); 1.80-1.70 (m, 4H, —CH₂—CH₂—); 1.35 (t, J=7.1 Hz, 3H, CH₃—CH₂—O—)

¹³C NMR (75 MHz; CDCl₃):

δ (ppm): 190.57; 66.22; 53.66; 44.15; 38.55; 28.22; 19.82; 14.20

UV spectrum:

λₘₐₓ=242 nm

Melting point: 58° C.

### EXAMPLE 15

### Oxidation of Sulforaphane

1.77 g of sulforaphane (1 equivalent) obtained in Example 4, as well as 15 mL of dichloromethane are introduced into the reactor, and the mixture is then degassed and set under nitrogen.

1.6 equivalents of meta-chloroperbenzoic acid solubilized in 25 mL of dichloromethane are placed in an isobaric dropping funnel and poured dropwise (within 20 mins) at room temperature, into the reactor. The addition causes exothermy for producing oxidized sulforaphane i.e. 1-isothiocyanato-4-(methylsulfonyl)butane or 4-methylsulfonylbutyl-isothiocyanate,

Stirring is maintained for 2 hours at room temperature. A white precipitate forms.

After this period, the reactor is cooled to −20° C. and maintained for 1 hour at this temperature before carrying out filtration.

The filtrate and the solid are analyzed by NMR. The solid contains derivatives of MCPBA, while the filtrate contains the oxidized product, as well as a few traces of aromatic residues and of residual sulforaphane.

The product is washed with a minimum of a solution saturated with NaHCO₃ in order to remove the benzoic acid, and then chromatographed on silica gel, eluent CH₂Cl₂ 100% and then AcOEtT 100%.

After evaporation of the solvents, 1.27 g of a yellowish liquid which solidifies are obtained with a yield of 66%.

TLC: Eluent: AcOEt 100%;

Developer: phosphomolybdic acid;

Rf=0.5.

¹H NMR (300 MHz; CDCl₃):

δ (ppm): 3.61 (t, 2H, —CH₂—NCS); 3.07 (t, 2H, —S(O)₂—CH₂); 2.94 (s, 3H, CH₃—S(O)₂—); 2.07-1.85 (m, 4H, —CH₂—CH₂—).

¹³C NMR (75 MHz; CDCl₃):

δ (ppm): 53.63; 44.44; 40.74; 28.55; 19.79.

UV spectrum:

λₘₐₓ=245 nm

Melting point: 58° C.

### EXAMPLE 16

### Synthesis of the Thiourea

### N-(4-(methylsulfonyl)butyl)morpholine-4-carbothioamide from the Oxidized Sulforaphane Obtained in Example 15

1-isothiocyanato-4-(methylsulfonyl)butane is placed in a reactor in presence of dichloromethane and of 1.2 equivalents of morpholine, and then the mixture is refluxed and maintained at this temperature for 1 hour. After removal of the solvent, the crude reaction mixture is purified by crystallization from ethyl acetate. The thiourea is thereby obtained pure as a white solid with a yield of 92% and has the following formula

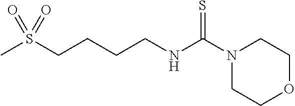 [SMILES: CS(=O)(=O)CCCCNC(=S)N1CCOCC1]

TLC:

Eluent: EtOAc; Developer; phosphomolybdic acid; Rf=0.3

¹H NMR (300 MHz; DMSO):

δ (ppm): 7.73 (broad peak, 1H, —NH—); 3.73 (m, 4H, —CH₂—O—CH₂—); 3.57 (m, 4H, CH₂—N—CH₂—); 3.53 (m, 2H, —CH₂—NH—C(═S)—); 3.12 (m, 2H, —CH₂—(SO₂)—); 2.94 (s, 3H, CH₃—(SO₂); 1.66 (m, 4H, —CH₂CH₂CH₂CH₂—)

¹³C NMR (75 MHz; DMSO):

δ (ppm): 181.70; 65.58; 53.14; 47.45; 44, 45; 27.24; 19.28

Melting point: 126° C.

UV spectrum

λₘₐₓ=221 nm et 242 nm

### EXAMPLE 17

### Synthesis of a Synthetic Thiourea, 1,3-bis(5-oxohexyl)thiourea

6-isothiocyanatohexan-2-one is placed in a reactor in the presence of water, and the mixture is refluxed and maintained at this temperature for 24 hours. After removal of the solvent, the crude reaction mixture is purified on a silica gel chromatographic column (CH₂Cl₂/MeOH). The thiourea is thus obtained pure as a white solid with a yield of 72% and has the following formula

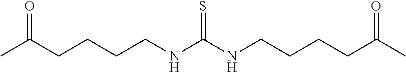 [SMILES: CC(=O)CCCCNC(=S)NCCCCC(C)=O]

TLC:

Eluent: CH₂Cl₂—MeOH 40/1; Developer: phosphomolybdic acid;

Rf=0.3

¹H NMR (300 MHz; CDCl₃):

δ (ppm): 6.10 (broad peak, 2H, —NH—); 3.44 (broad peak, 4H, —CH₂NH—); 2.50 (m, 4H, CH₂—(C═O)—); 2.15 (s, 6H, CH₃—(C═O)—); 1.61 (m, 8H, —CH₂—CH₂)

¹³C NMR (75 MHz; CDCl₃):

δ (ppm): 209.00; 181.55; 43.86; 42.80, 30.00; 28.21; 20.34

Melting point: 53° C.

UV SPECTRUM

λₘₐₓ=242 nm

### EXAMPLE 18

### Synthesis of a Synthetic Thiourea 1-(4-(methylsulfinyl)butyl)-3-(5-oxohexyl)thiourea

6-isothiocyanatohexan-2-one is placed in a reactor in the presence of dichloromethane and of 2 equivalents of 4-methylsulfinylbutanamine, the amine precursor of the sulforaphane obtained in Example 3, and then the mixture is refluxed and maintained at this temperature for 3 hours. After removal of the solvent, the crude reaction mixture is purified on a silica gel chromatographic column (CH₂Cl₂/MeOH). The thiourea is thus obtained pure as a pale yellow and viscous oil with a yield of 74%.

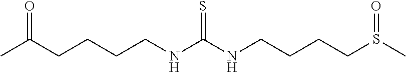 [SMILES: CC(=O)CCCCNC(=S)NCCCCS(C)=O]

TLC:

Eluent: CH₂Cl₂—MeOH 9/1; Developer: phosphomolybdic acid; Rf=0.5

¹H NMR (300 MHz; CDCl₃):

δ (ppm): 6.67 (broad peak, 2H, —NH—); 3.55 (2H, —CH₂—NH—); 3.44 (2H, —CH₂—NH—); 2.75 (t, 2H, J=7.2 Hz, —CH₂ (S═O)—); 2.59 (s, 3H, —CH₃(S═O)—); 2.48 (t, 2H, J=6.6 Hz, —CH₂(C═O)—); 2.48 (s, 3H, —CH₃(C═O)—); 1.75 and 1.57 (m, 8H, —CH₂—CH₂—)

¹³C NMR (75 MHz; CDCl₃):

δ (ppm): 209.02; 182.12; 53.51; 43.87; 43.44; 42.87; 38.55; 30.01; 28.34; 28.00; 20.55; 20.07

UV SPECTRUM

λₘₐₓ=242 nm

### EXAMPLE 19

### Synthesis of a Synthetic Thiourea by Coupling with 6-isothiocyanatohexan-2-one and Piperidine

6-isothiocyanatohexan-2-one is placed in a reactor in the presence of dichloromethane and of 1.2 equivalents of piperidine, and then the mixture is refluxed and maintained at this temperature for 1 hour. After removal of the solvent, the crude reaction mixture is purified on a silica gel chromatographic column (CH₂Cl₂/MeOH). The thiourea is thereby obtained pure as a viscous oil with a yield of 93% and has the following formula:

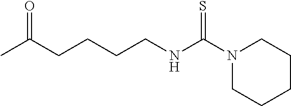 [SMILES: CC(=O)CCCCNC(=S)N1CCCCC1]

TLC:

Eluent: CH₂Cl₂—MeOH 40/1; Developer: phosphomolybdic acid;

Rf=0.5

¹H NMR (300 MHz; CDCl₃):

δ (ppm): 5.88 (broad peak, 1H, —NH—); 3.80 (m, 4H, N—CH₂); 3.63 (m, 2H, CH₂NH—); 2.51 (m, 2H, —CH₂(C═O)—); 2.15 (s, 3H, CH₃—(C═O)—); 1.62 (m, 10H, —CH₂CH₂CH₂— and 3 CH₂ ring)

¹³C NMR (75 MHz; CDCl₃):

δ (ppm): 209.43; 181.09; 48.68; 45.39; 42.84; 30.04; 28.40; 25.39; 24.29; 20.05

UV SPECTRUM

λₘₐₓ=216 nm, 248 nm

### EXAMPLE 20

### Synthesis of methyl-N-(5-oxohexyl)-carbamodithioate

2 equivalents of sodium methanethiolate are solubilized in DMF. The mixture is cooled to 0° C. and 2 equivalents of 37% HCl are added. The mixture is maintained for 1 hour at this temperature and then 1 equivalent of 6-isothiocyanatohexan-2-one is added. The medium is maintained at this temperature for 3 hours and then for a few hours at room temperature. After extraction with dichloromethane and washing with the acid, the organic phases are collected, dried and the solvent is evaporated. The obtained product is then purified on a silica gel chromatographic column (CH₂Cl₂). The pure product is obtained as a white solid with a yield of 45% and has the following formula:

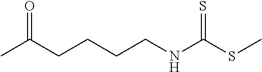

TLC:

Eluent: CH₂Cl₂—MeOH 99/1; Developer: phosphomolybdic acid; Rf=0.4

¹H NMR (300 MHz; CDCl₃):

δ (ppm): 7.32 (broad peak, 1H, —NH—); 3.71 (m, 2H, —CH₂NH—); 2.63 (s, 3H, —S—CH₃); 2.51 (m, 2H, —CH₂—(C═O)); 2.15 (s, 3H, CH₃—(C═O)—); 1.65 (m, 4H, —CH₂CH₂CH₂CH₂—)

Tautomer (minority):

δ (ppm): 7.75 (broad peak, 1H, —NH—); 3.43 (m, 2H, —CH₂NH); 2.67 (s, 3H, —S—CH₃); 2.51 (m, 2H; —CH₂—(C═O)); 2.15 (s, 3H, CH₃—(C═O)); 1.65 (m, 4H, —CH₂CH₂CH₂CH₂)

¹³C NMR (75 MHz; CDCl₃):

δ (ppm): 208.94; 198.96; 46.70; 42.78; 30.01; 27.51; 20.37; 18.06

Melting point: 52° C.

UV spectrum:

λₘₐₓ=222 nm; 253 nm; 270 nm

### EXAMPLE 21

### Synthesis of 4-methylthiobutylisothiocyanate

Examples 1 and 2 are reproduced and then Example 4 was directly made. This means that this isothiocyanate is synthesized according to the same method as sulforaphane, by reaction of the corresponding amine (4-methylthiobutanamine) in the presence of carbon disulfide and of di-tert-butyl dicarbonate.

The amine is obtained by following the first steps of the synthesis of sulforaphane described in this patent, except for the oxidization step.

4-methylthiobutanamine reacts in the ethanol in the presence of 1 equivalent of triethylamine and of 10 equivalents of carbon disulfide. After reaction, the mixture is cooled to 0° C. before adding thereto 0.99 equivalents of di-tert-butyl dicarbonate and 3 mol % of DMAP in solution in ethanol. After returning to room temperature and a further 2 hours of reaction, the reaction medium may be treated: removal of the solvent, washing with a hydrochloric acid solution, decantation and removal of the solvent. Additional purification on a silica gel chromatographic column may optionally be performed. The isothiocyanate is obtained as a yellow liquid with a yield of 92% for this step and has the following formula:

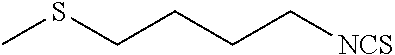

The overall yield of the reaction, including the synthesis of the amine, is 92%.

TLC:

Eluent: Hexane —CH₂Cl₂ 2/1: Developer: phosphomolybdic acid;

Rf=0.3

¹H NMR (300 MHz; CDCl₃):

δ (ppm): 3.55 (t, 2H, J=6.4 Hz, —CH₂NCS); 2.53 (t, 2H, J=6.8 Hz, —CH₂SCH₃); 2.10 (s, 3H, 9CH₂SCH₃); 1.67-1.87 (m, 4H, —CH₂CH₂)

¹³C NMR (75 MHz; CDCl₃):

δ (ppm): 44.89; 33.26; 28.10; 28.8; 15.4

Refractive index: n_{D}²⁰=1.5278

UV SPECTRUM

λₘₐₓ=243 nm

### EXAMPLE 22

### Synthesis of the Thiourea Derived from the Isothiocyanate According to Example 21, 1,3-bis(4-methylthio)-butyl)thiourea

The 4-methylthiobutylisothiocyanate obtained in Example 22 is placed in a reactor in the presence of dichloromethane and of 1.2 equivalents of 4-methylthiobutanamine, and the mixture is then refluxed and maintained at this temperature for 2 hours. After removal of the solvent, the crude reaction product is purified on a silica gel chromatographic column (CH₂Cl₂/MeOH). The thiourea is thus obtained pure as a solid with a yield of 89% and has the following formula:

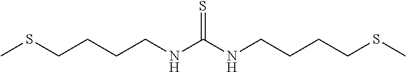

TLC:

Eluent: CH₂Cl₂—MeOH 99/1; Developer: phosphomolybdic acid;

Rf=0.2

¹H NMR (300 MHz; CDCl₃):

δ (ppm): 5.86 (broad peak, 2H, —NH—); 3.44 (m, 4H, CH₂—NH—); 2.54 (t, 4H, J=6.8 Hz; —CH₂—S—); 2.09 (s, 6H, CH₃—S—); 1.70 (m, 8H, —CH₂CH₂CH₂CH₂—)

¹³C NMR (75 MHz; CDCl₃):

δ (ppm): 181.15; 43.48; 33.23; 27.36; 25.57; 15.04

Melting point: 46° C.

UV SPECTRUM

λₘₐₓ=243 nm

### EXAMPLE 23

### Synthesis of (4-methylsulfinyl-1-(S-methyl-dithio-carbamyl)-butane sulforamate

5 equivalents of sodium methanethiolate are solubilized in DMF. The mixture is cooled to 0° C. and 5 equivalents of 37% HCl are added. The mixture is maintained for 1 hour at this temperature, and then 1 equivalent of sulforaphane obtained according to Example 4 is added. The medium is maintained with stirring, then the temperature is left to rise up to room temperature. At the end of the reaction, after extraction with dichloromethane and washing with the acid, the organic phases are collected, dried and the solvent is evaporated. The obtained product is then crystallized from ethyl acetate. The pure product is obtained as a whitish solid and has the following formula:

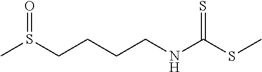

TLC:

Eluent: EtQAc/MeOH 40/2; Developer: phosphomolybdic acid; Rf=0.3

¹H NMR (300 MHz; CDCl₃):

Sulforamate:

δ (ppm): 8.26 (a large, 1H, —CH₂—NH—C(S)—); 3.77 (m, 2H, —CH₂—NH—); 2.75 (m, 2H, —S(O)—CH₂—); 2.59 (s, 6H, CH₃—S(O)— and —S—CH₃); 1.85 (m, 4H, —CH₂CH₂—)

Tautomer (minority):

8.40 (broad s, 1H, —SH); 3.48 (m, 2H, —CH₂—NH—); 2.76 (m, 2H, —S(O)—CH₂); 2.64 (s, 6H, CH₃S(O)— and —S—CH₃); 2.59 1.85 (m, 4H, —CH₂—CH₂—)

¹³C NMR (75 MHz; CDCl₃):

Sulforamate:

δ (ppm): 199.03; 5336; 46.26; 38.49; 27.13; 20.16; 18.00

Melting point: 96° C.

UV Spectrum: λₘₐₓ=250 and 272 nm

### EXAMPLE 24

### Depigmentation Efficiency of the Synthesized Compounds

Various synthesized molecules were tested in order to determine their depigmenting power. The idea was to evaluate the effect of each of them on the production of melanin by melanocytes maintained in a culture medium.

For these tests, 2 lines of melanocytes were cultivated, human melanocytes of the SKMel type on the one hand and a murine line of the B16F10 type on the other hand. Here, we only show the results obtained on the human line.

The melanin content was determined by spectrophotometry, after 3 days of incubation in the presence of the molecule to be tested and cell lysis.

Kojic acid, a well-known depigmenting agent is used as a reference for making a hierarchy of the results. The results are shown in Table 2 which shows the percentage of residual pigmentation which is calculated as the ratio of the amount of melanin produced in the presence of the active ingredient over the amount of melanin produced in a control culture (cells alone) on the one hand and the relative activity of the relevant active substance with respect to that of kojic acid at the same concentration, on the other hand.

For each measurement, the analyses were repeated. The indicated value corresponds to the calculated average. When the experiments are repeated, the average variation coefficient of the residual pigmentation percentage is of the order of 3% (comprised between 1.5% and 6% (for 3 tests).

Table 2 shows the results obtained on the human line SKMel for a set of typical molecules with which it is possible to cover the whole of the families and chemical modifications proposed in the patent from the basic molecule (sulforaphane, other derived isothiocyanates, oxidized forms, symmetrical thioureas, other thioureas, derivatives from addition of nucleophilic agents on the isothiocyanate function of sulforaphane, . . . ). This list is neither exhaustive nor limiting and the same type of results was obtained for the other molecules.

Moreover, the basic amines, constituents of the thioureas, were also evaluated, which allows us to demonstrate that our structures actually provide added value, in terms of depigmenting power, with respect to the simple amines which make them up.

For the whole of the molecules, we also evaluated their cytotoxicity by the MTT test. It notably emerges from this test that the thioureas shown here are atoxic (IC₅₀>100 μM). They may therefore be used at concentrations much greater than the 5 μM taken here as a reference without any risk for cell viability. As an example, when the kojic acid concentration is increased from 5 to 100 μM, the residual pigmentation percentage passed from 97 to 73%. The same experiment conducted on 1,3-bis-(4-(methanesulfinyl)butyl)-thiourea, causes a variation in the residual pigmentation which passes from 20 to 14%. However, the molecule is here already very efficient from the lowest concentration upwards.

TABLE 2

| | | | Resid. | |
|---|---|---|---|---|
| | | | Pig. | Eff./Ref. |
| Family | Molecule | Cc (μM) | (%) | to 5 μM |
| Ref.: kojic acid | 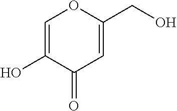 | 5 | 97 | 1 |
| Isothiocyanates | 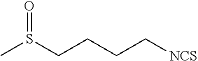 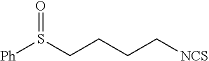 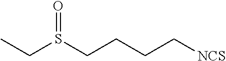 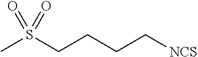 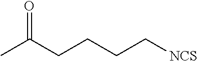 | 5 5 5 5 5 | 34 71 63 54 84 | 22 10 12 15 5 |
| Other derivative | 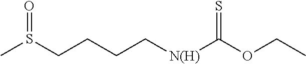 | 5 | 57 | 14 |
| Thioureas | 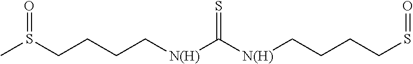 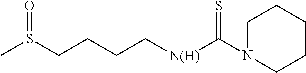 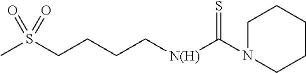 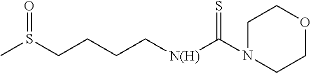 | 5 5 5 5 | 20 24 21 49 | 27 25 23 17 |
| Basic amines | 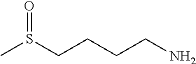 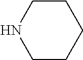 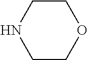 | 5 5 5 | 67 50 86 | 11 17 5 |
| Thioureas | 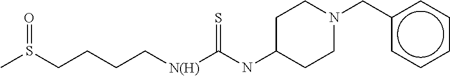 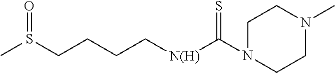 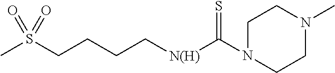 | 5 5 5 | 60 59 55 | 13 14 15 |

It is quite understood that the invention is by no means limited to the embodiments described above and that may modifications may be made thereto without departing from the scope of the appended claims.

1. A method for synthesizing an isothiocyanate of general formula (I)
SCN—R₁—R₄—R₂ (I)
wherein R₁ and R₂ represent independently of each other an alkyl, aryl or alkylaryl group, R₄ represents a carbonyl, sulfinyl, sulfonyl, sulfide group and of its derivatives comprising a step for reaction of an amine having the general formula (II)
NH₂—R₁—R₄—R₂ (II)
wherein R₁ and R₂ represent independently of each other an alkyl, aryl or alkylaryl group, R₄ represents a carbonyl, sulfinyl, sulfonyl or sulfide group in the presence of carbon sulfide and of di-tert-butyl dicarbonate with formation of the corresponding aforesaid isothiocyanate.

2. The method according to claim 1, wherein said group R₄ represents a sulfinyl group and wherein said amine is an alkylsulfinylalkylamine.

3. The method according to claim 2, wherein said alkylsulfinylalkylamine is obtained by oxidation of alkylthioalkylamine in solution in a solvent based on trifluoroethanol.

4. The method according to claim 1, wherein said amine of general formula (II) is 4-methylsulfinylbutylamine and said corresponding formed isothiocyanate is sulforaphane.

5. The method according to claim 1, wherein R₄ represents a carbonyl group and wherein said amine comprises a keto group.

6. The method according to claim 5, wherein said amine comprising a keto group is 4-methylketobutylamine and the formed corresponding isothiocyanate is 6-isothiocyanatohexan-2-one.

7. The method according claim 1, comprising a reaction of said corresponding isothiocyanate of general formula (I) with a primary or secondary amine for forming a thiourea.

8. The method according to claim 7, wherein the amine is a primary amine having the general formula HNR₅R₆ wherein R₅ represents a hydrogen atom and R₆ a methylsulfinylbutyl group.

9. The method according to claim 7, wherein the amine is a primary amine having the general formula HNR₅R₆ wherein R₅ represents a hydrogen atom and R₆ a linear, cyclic or branched alkyl, alkenyl, alkylaryl, aryl, alkynyl group optionally including one or more heteroatoms.

10. The method according to claim 7, wherein the amine is a secondary amine having the general formula HNR₅R₆ wherein R₅ and R₆ represent independently of each other a linear, cyclic or branched alkyl, alkenyl, alkylaryl, alkynl group optionally including one or more heteroatoms.

11. The method according to claim 1, comprising a reaction between said isothiocyanate of general formula (I) and a nucleophilic agent, in particular an alcohol or a thiol.

12. The method according to claim 2, comprising oxidation of the sulfinyl radical into a sulfonyl radical, by adding an oxidizing agent, in particular perbenzoic acid or one of its halogenated derivatives.

13. A synthetic and isolated compound of general formula (III) obtained by the method according to claim 1,
R₇—R₁—R₄—R₂ (III)
wherein R₄ is a carbonyl, sulfinyl or sulfonyl, sulfide group, R₇ represents an amino, isothiocyanato group, a group of the —NH—C(═S)—R′ type, wherein R′ is of the alcoholic (-DR″), thiolate (SR″), amino (—NR″R′″) type and R₁ and R₂ both represent independently of each other an alkyl, aryl or alkylaryl group.

14. The use of a compound according to claim 13, as a depigmenting agent or in the treatment or in the treatment of hyper pigmentation.

15. The use of a compound according to claim 13, as an inductor of enzymes of phase II, as an inhibitor or moderator of phase I enzymes in anti-cancer treatment, in the protection of the skin against effects of pollution.

16. The use of a compound according to claim 13, in the treatment of canities, in the protection of the skin against UV radiations or other radiations and effects of the latter, for example in phototherapies, radiotherapies, exposures to the sun, at erythemas, damages to DNA, repair, signaling and cancerisation of the skin in the treatment of lucite, in the treatment of the inflammation, in particular of atopic dermatitis.
